(19) United States
(12) Patent Application Publication
(10) Pub. No.: US 2023/0197293 A1
(43) Pub. Date: Jun. 22, 2023

(54) SYSTEM AND METHOD FOR COMMUNICATIONS BETWEEN PATIENTS AND MENTAL HEALTH PROVIDERS

(71) Applicant: Morgan State University, Baltimore, MD (US)

(72) Inventors: Dawn Thurman, Bowie, MD (US); Rhonda Wells Wilbon, Columbia, MD (US)

(21) Appl. No.: 18/069,539

(22) Filed: Dec. 21, 2022

Related U.S. Application Data
(60) Provisional application No. 63/292,976, filed on Dec. 22, 2021.

(51) Int. Cl. G16H 80/00; G16H 10/20; G16H 50/20

(52) U.S. Cl. CPC G16H 80/00; G16H 10/20; G16H 50/20

(57) ABSTRACT

A computer-implemented method and system for recording, storing and analyzing patient information, for making automated healthcare treatment recommendations based on automated assessment of patient information, make healthcare provider recommendations to the patient, connect patient and healthcare provider online and facilitate and/or manage communication therebetween, record healthcare provider notes, recommendations and prescriptions, provide patient access to such healthcare provider notes, recommendations and prescriptions, and use anonymized data collected from the system for healthcare improvement research.

### BACKGROUND OF THE INVENTION

### Field of the Invention

The present invention relates to methods and systems for improving access to healthcare, and particularly mental healthcare.

### SUMMARY OF THE INVENTION

The invention is a computer implemented system and method that provides patients a convenient and user-friendly medium for eliciting, recording, maintaining, tracking and analyzing various health related information, questions, and symptoms, including thoughts and feeling which are particularly important for mental health patients, for making immediate healthcare recommendations to the patient based on the recorded information, for making healthcare provider recommendations based on the recorded information, for connecting patients online with appropriate healthcare providers, and for communicating the recorded information to healthcare providers for the development of appropriate treatment and therapy regimen, for recording healthcare provider notes and recommendations so that the patient can access them at their convenience, and for collecting anonymized data for research purposes. The patient's interface may be presented by a mobile app on a user's mobile device, on a laptop or desktop webpage, or combination of both. Similarly, healthcare providers can likewise access the healthcare provider side of the invention on a provider mobile app and/or website. A platform is proposed that allows for communications between patients and doctors/social workers/therapists/supervisors (providers).

In short, the invention provides various tools that a patient can readily access to record and assess important information concerning their health, including mental health, make healthcare recommendations based on that information, and immediately connect patients with healthcare providers, including therapists, doctors and social workers, and with emergency contacts, as in the case of a suicide hotline, for example. In particular, the invention provides various questionnaires, including a plurality of different mental health-focused questionnaires and surveys designed to probe the patient's mental state and sense of well-being, provides the patient various modality for recording thoughts, moods and feelings, including diary/journal interfaces, including textual and image-based input tools (emoticons, drawing boards, etc.), and a medical history module configured to elicit information from the patient and use it to build a genogram. A particular advantage of the invention is that the information input by the patient is continually analyzed using algorithms that assess the risk to the patient. According to one embodiment, the system may be configured to immediately alert a patient emergency contact and/or healthcare provider in the event the assessment meets a certain threshold. According to one option, the invention may make support group recommendations, connect and organize patients into support groups and manage online/virtual support group audio and/or video communications. According to one option, the patient may have the option to raise or lower that threshold, below a certain “may not exceed” level. In this way, the patient has a “constant listener” that is always “listening” to the patient's thoughts and making constant assessments concerning whether the patient is in immediate danger and/or needs immediate professional care.

Another advantage of the invention is that healthcare providers that are connected with patients using the invention have immediate access (subject to patient approval) to what may possibly be a large volume of patient-reported data and information concerning the patient's present and historical health and sense of well-being.

Another advantage of the invention is that the patient likewise has immediate access to healthcare provider notes and recommendations for treatment and medication.

Another advantage of the invention is that it can provide a source of anonymized original patient data, coupled with healthcare provided data, that can be used for research for more detailed, larger-scale analysis, or to develop improved healthcare services.

While the invention is described primarily from the standpoint of mental healthcare, it may be adapted for any type of healthcare.

### BRIEF DESCRIPTION OF THE DRAWINGS

The foregoing summary, as well as the following detailed description of the preferred invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a representation of the users and elements of the invention and the conceptual lines of communication between them.

FIG. 2 is a screen shot of a Wellness Measures and Assessments introduction webpage according to an embodiment of the invention.

FIG. 3 is a screen shot of an Assessments webpage according to an embodiment of the invention, including tabs for selection of three different health and wellness assessments.

FIG. 4 is a screen shot of a DASS-21 Depression, Anxiety, and Stress Scale assessment webpage according to an embodiment of the invention.

FIG. 5 is a screen shot of a Patient Health Questionnaire webpage according to an embodiment of the invention.

FIG. 6 is a screen shot of an Edinburgh Postnatal Depression Scale assessment webpage according to an embodiment of the invention.

FIG. 7 is a screen shot of a Crisis Support webpage according to an embodiment of the invention, including a 911 button for immediate access to 911 emergency services.

FIG. 8 is a screen shot of a journal and vision board introduction webpage according to an embodiment of the invention.

FIG. 9 is a screen shot of a Vision Board creation webpage according to an embodiment of the invention.

FIG. 10 is a screen shot of a Support Circle webpage according to an embodiment of the invention where users can connect with members of a support group.

FIG. 11 is a screen shot of a Mindfulness introduction webpage according to an embodiment of the invention where users can select various calming, relaxation and/or meditation exercises.

FIG. 12 is a screen shot of a My Provider webpage according to an embodiment of the invention.

FIG. 13 depicts a computing node of a system according to an embodiment of the present invention.

FIG. 14 depicts a computing environment according to an embodiment of the present invention.

FIG. 15 depicts abstraction model layers according to an embodiment of the invention.

### DETAILED DESCRIPTION OF THE INVENTION

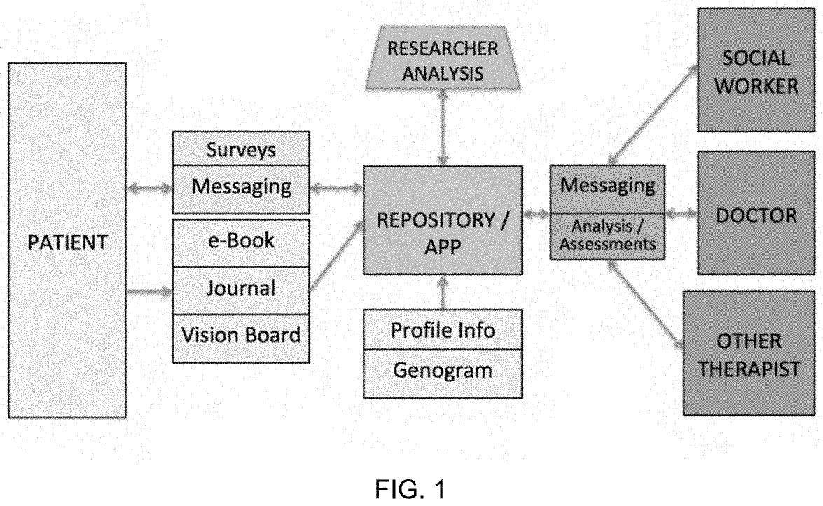

The present invention is a computer-implemented platform that provides for and supports direct and immediate communications between patients and doctors/social workers/therapists/supervisors (providers). The invention provides a medium for capturing patient information, including family medical and mental health history, patient medical and mental health history and contemporaneous patient mental health condition, symptoms and/or sense of well-being, and to provide automated algorithm-based preliminary diagnoses and treatment recommendations. See, e.g., FIG. 1. The invention also enables mental health patients to interact with healthcare providers when a physical appointment is not possible or available. While it is hoped that the patient-user will take advantage of the opportunity to connect with healthcare providers using the invention, the patient can use the platform and the tools thereon without requiring communications between patient and provider.

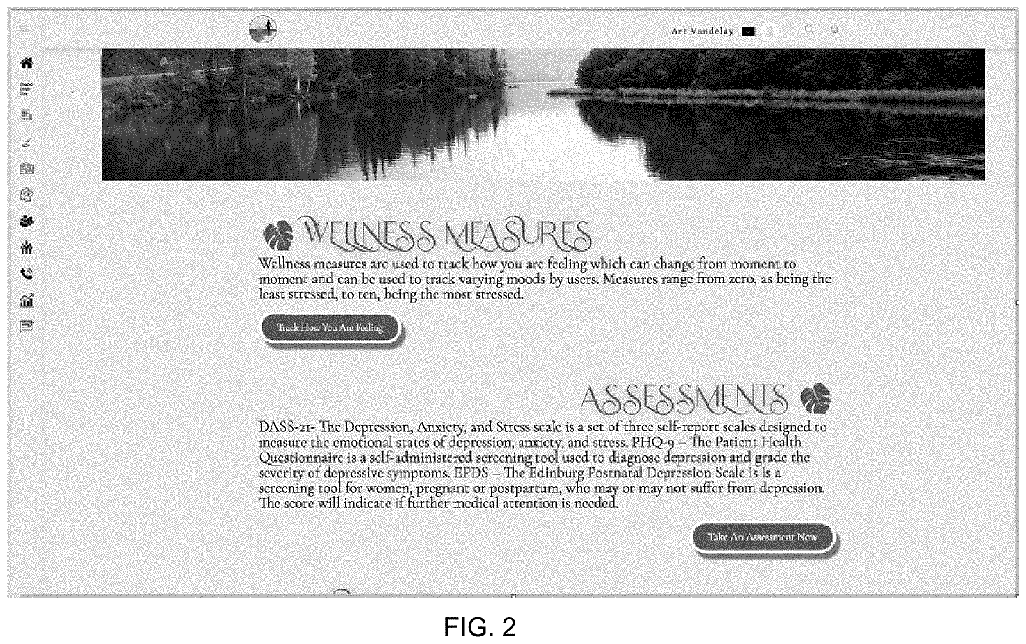

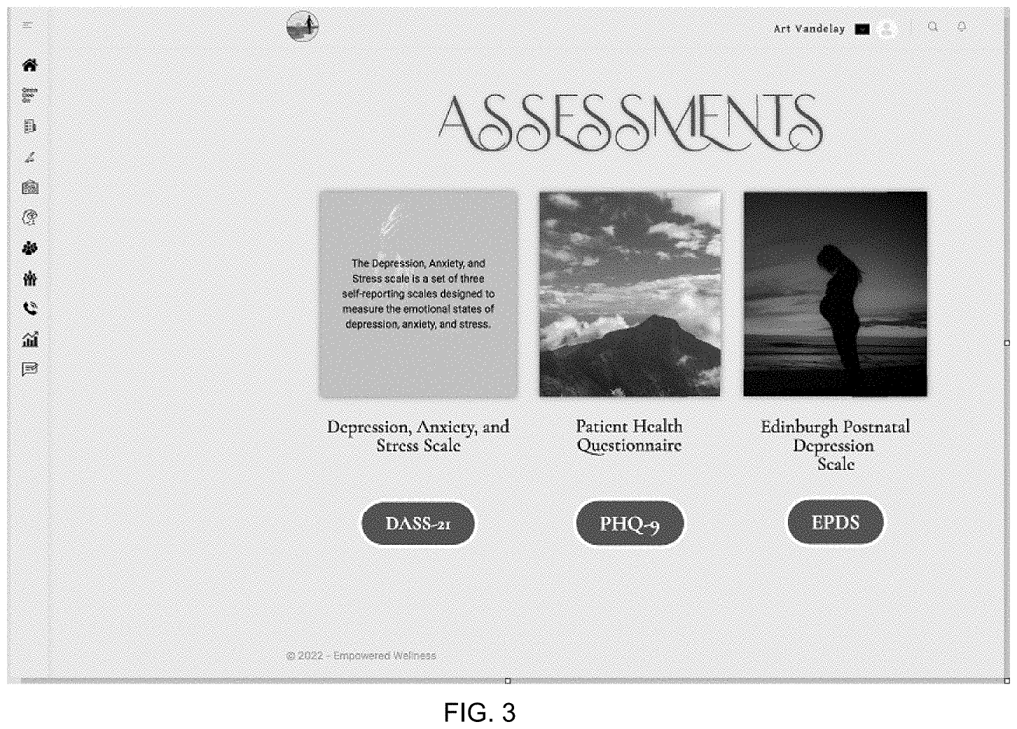

The invention features comfortable, calming and user-friendly interface with easy navigation between different functionalities, see, e.g., FIGS. 2-12. Each page features the main menu in icon/pictograph form, representing, for example, an assessment page, a calendar/appointment page, a notes page, a vision board page, a mindfulness page, a support circle page, a provider page, an emergency contact page, a progress/charting page, and a chat/messaging page.

According to various embodiments, tools on the assessment page of platform may include electronic surveys that gauge the patient's mood and/or health at a particular instance. See, e.g., FIGS. 2-6. Such surveys may include various measures, such as stress, anxiety, and depression. According to various embodiments, the patient may be prompted to select answers based on a 0-10 scale, for example, where 0 is excellent and 10 indicates a severe state, or the patient may be prompted to select from more qualitative answers, such as various degrees of agree or disagree, or always, sometimes and never.

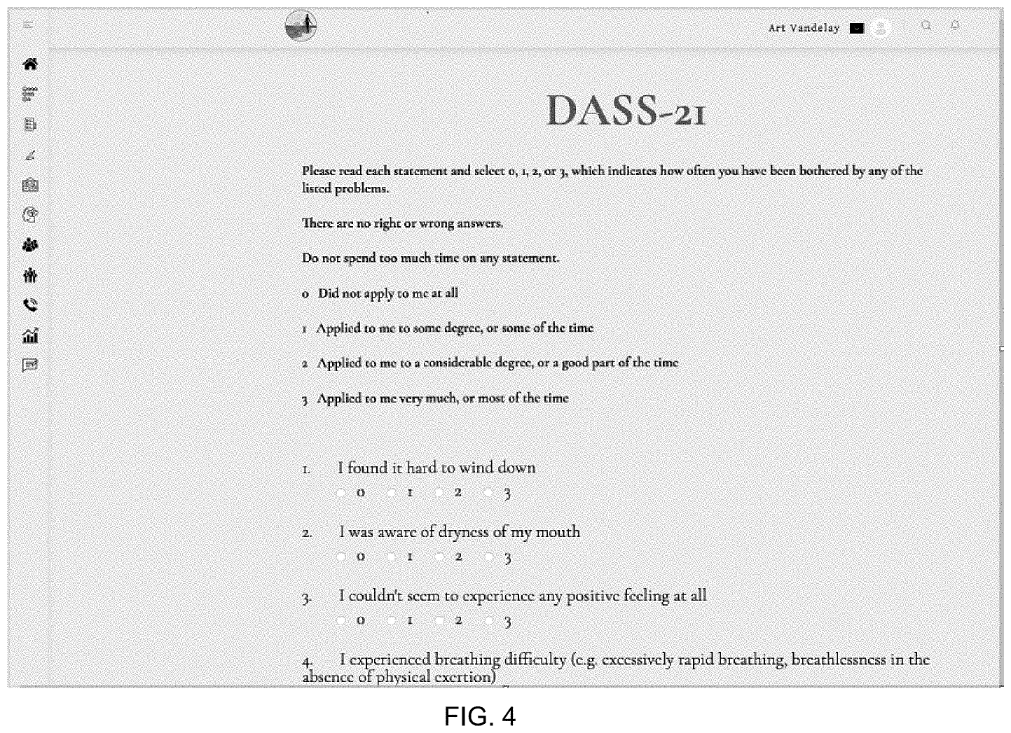

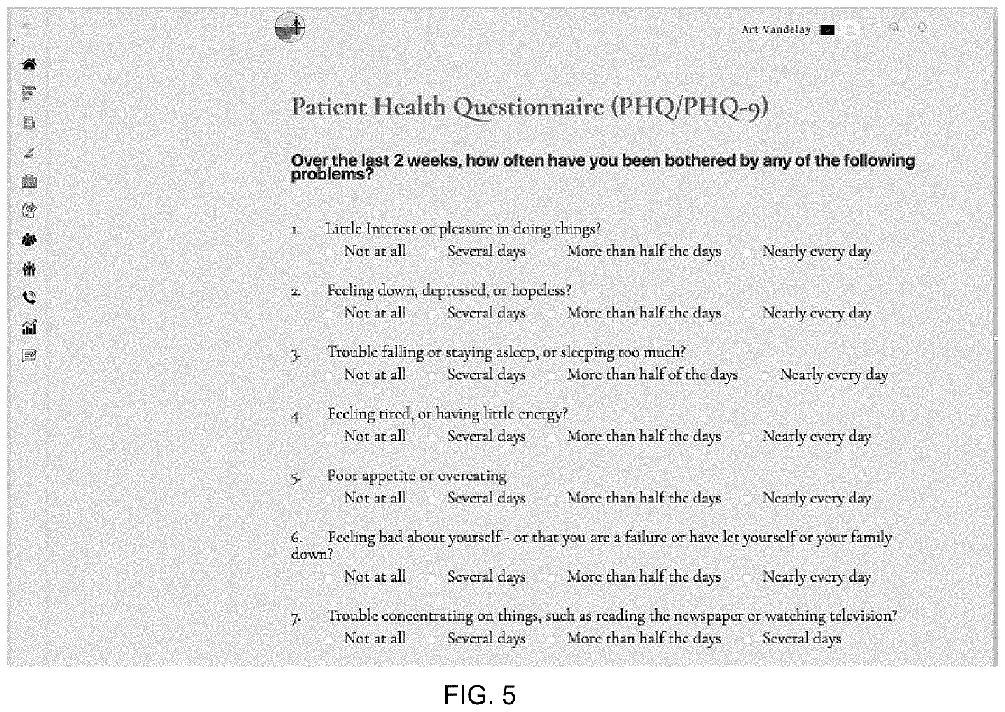

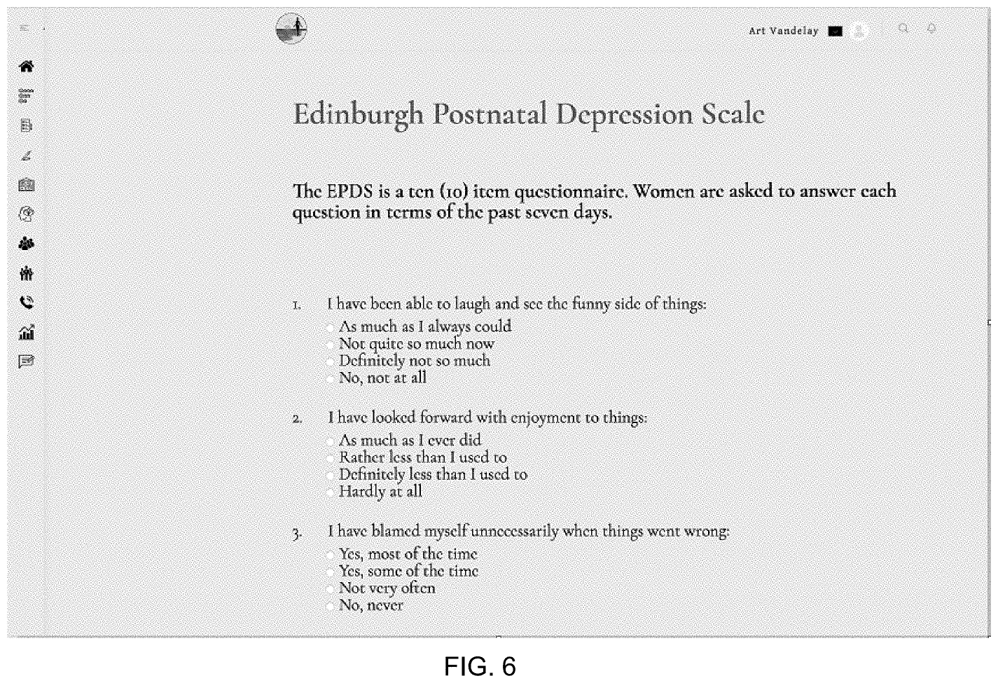

Other platform tools may include assessments that gauge the patient's sense of well-being, including DASS (FIG. 4), PHQ/PHQ-9 (FIG. 5), MDQ, DSM-5 (PCL-5), and EPDS (FIG. 6), among others. The system may be configured to provide instant assessments based on the patient's answers and may suggest, for example, that the patient may be suffering from mild or severe depression.

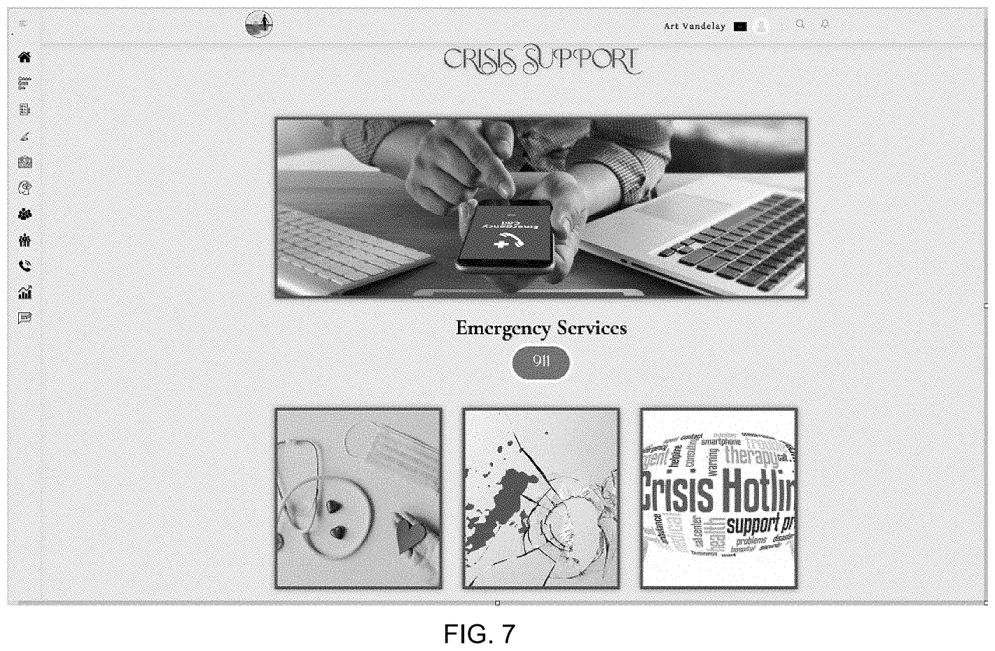

According to a preferred embodiment, the system may be configured to allow the user to connect directly to an emergency contact, emergency services (911, for example, see FIG. 7.), a crisis hotline, or mental health professional. According to a more preferred embodiment, the system may be configured to automatically connect to emergency services, a crisis hotline, an emergency contact and/or mental health professional, based on patent assessments that meet certain pre-determined thresholds. Such pre-determined thresholds may be set by the system. According to various embodiments, the pre-determined thresholds may be fixed or they may be adjustable by the user based on the user's perceived sensitivities to various inputs and life conditions.

According to another optional feature of the invention, the patient may be prompted to input information concerning family relationships and medical history, including mental health, that the system uses to build a genogram which may be accessed by patient and (upon patient authorization) healthcare provider. According to various embodiments, the genogram may be generated by the invention or generated by third party software.

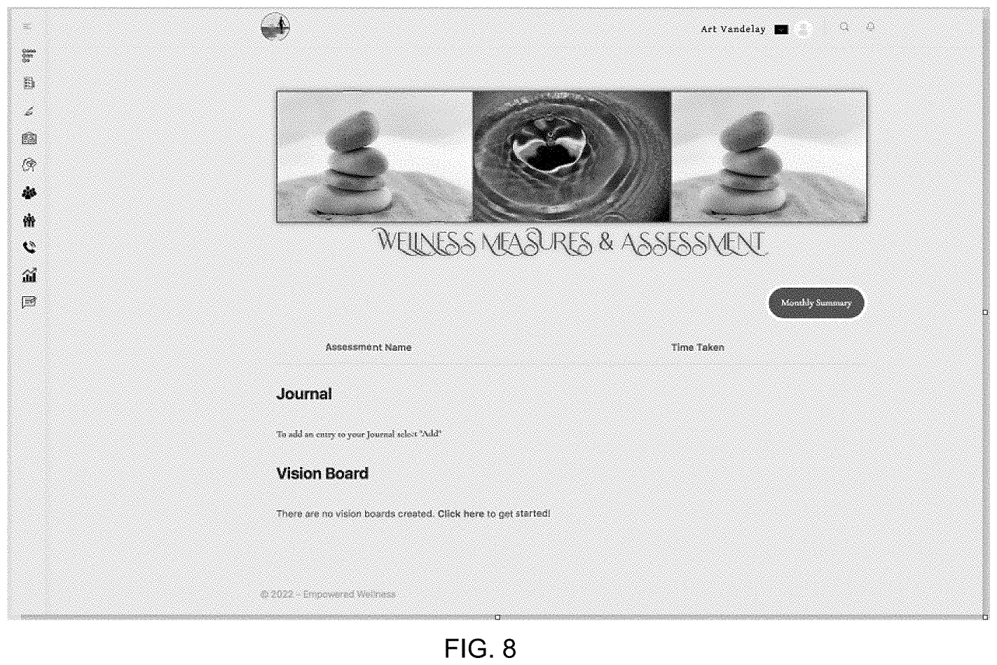

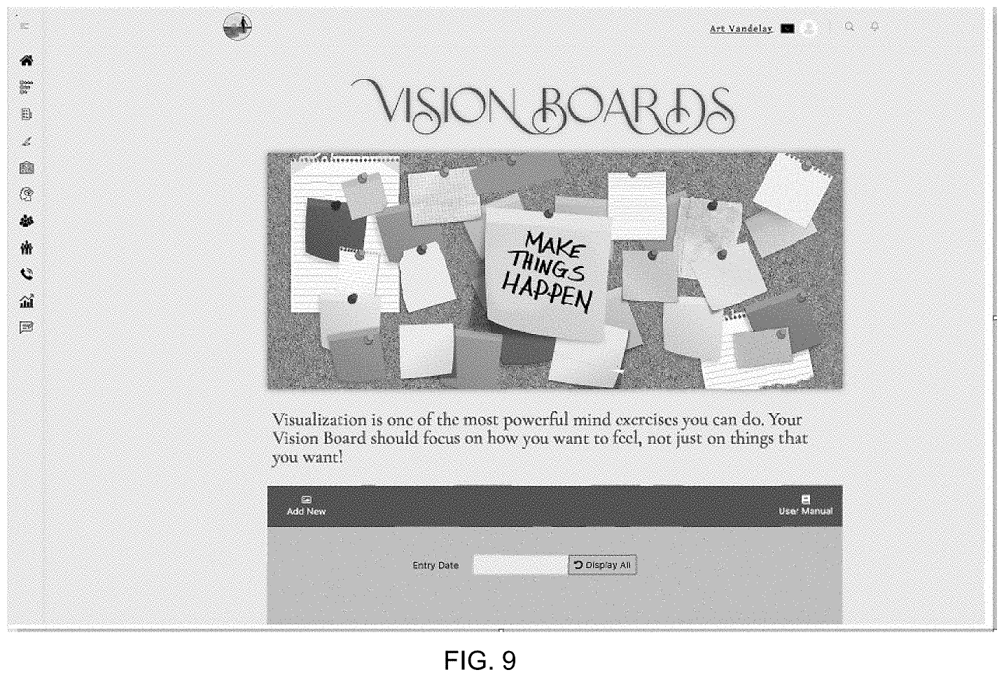

According to further features of the invention, the patient is provided with various media to enter notes in the system, including by e-book or by physically taking notes on paper (journal). The invention may also provide an input medium according to which the patient may create a vision board (FIGS. 8 and 9), which involves conveying pictorially how he/she is feeling at the moment. Any notes prepared offline (not entered directly into the system) may be uploaded into the platform, and if appropriate, be subject to optical character recognition (OCR) for digital transcription.

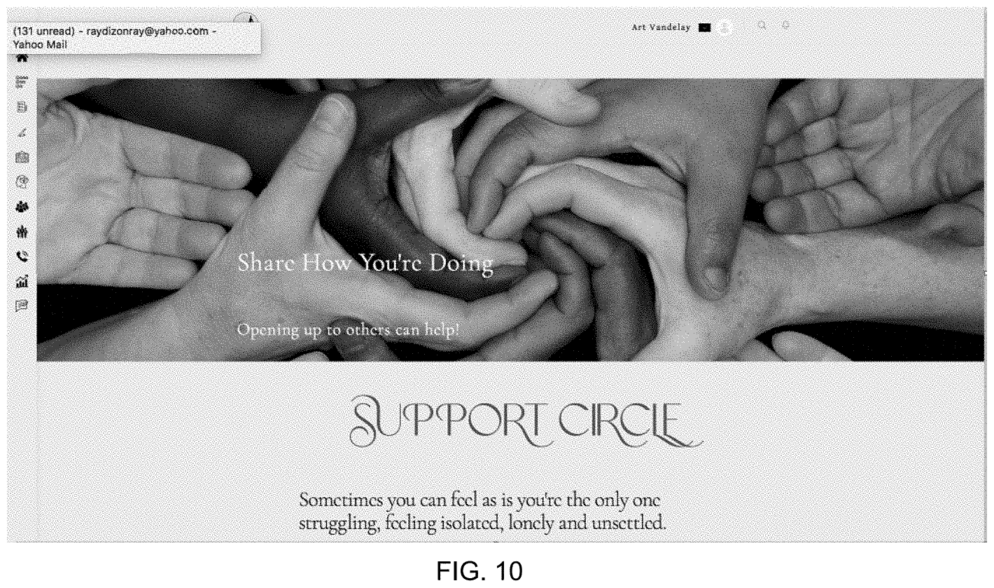

The invention may also provide patients with opportunities to connect with support groups (FIG. 10), including suggesting that various patient users join a particular support group, organizing such support group, and facilitating and/or managing virtual and/or in-person support group meetings. A patient may optionally elect to allow the system to share selected patient information with members of a support group.

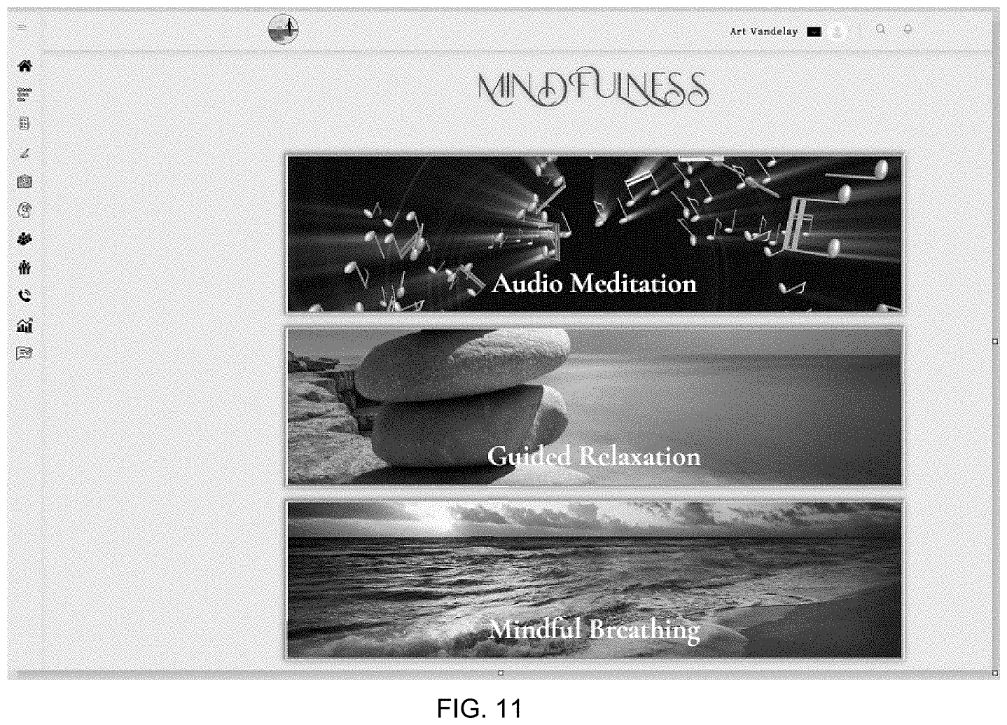

According to another embodiment of the invention, there may be provided a mindfulness page which the user may user to help with calming, relaxation, and/or meditation (FIG. 11).

One significant advantage of the invention is that the patient is able to access daily entries and statistics, which registers how many times that person has entered into the account, how many times a patient's mood has been tested, how many times a survey has been accessed for assessment, how many times a patient has entered data into a journal or e-book, vision board, or genogram, and how many times the patient has reached out to a support circle. The patient can also view graphical displays that depict activity in a respective category over a defined period of time. According to this feature of the invention, a patient or authorized provider can see and track a patient's progress and changes over time, including the ability to connect progress and/or changes to various patient life events.

Healthcare providers that may be accorded access to patients and patient information according to the invention may be any one or more of a doctor, social worker, therapist or other person who is qualified to diagnose and treat mental health patients. Subject to patient authorization, any one or more healthcare providers may have access to and analyze the patient notes and information and communicate with the patient using the invention concerning method(s) of possible treatment or to recommend an in-person appointment. Any notes, assessments, recommendations, etc. by the healthcare provider may be saved in the invention and accessed by the patient.

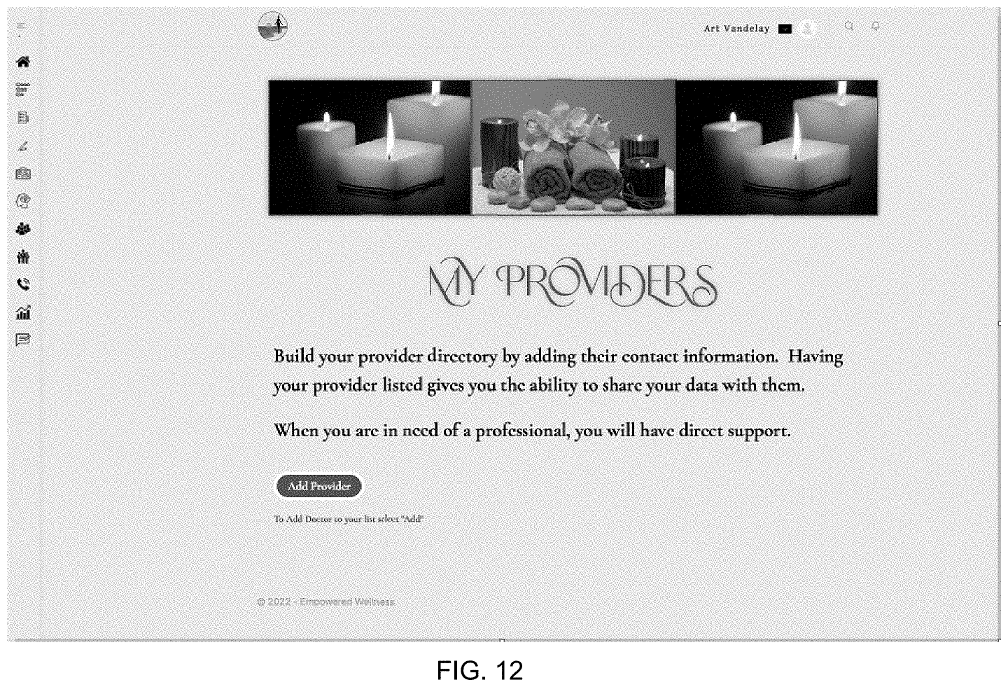

Providers and patients can be matched up if patients do not currently deal with a mental health professional and want to engage with one or more providers (FIG. 12). Providers have the ability to mention any specific specialties that they have that could benefit patients.

Further features of the invention include software that will sequence journal entries and other items in chronological order, allow for messaging among providers and patient, allow for training, provide profile information, house survey instruments and conduct assessments, accommodate vision boards and genograms that show family relationship dynamics and patterns of behavior, presenting providers with a wealth of historical and current data, and effectively guiding providers towards possible solutions for their patients, while enabling multiple providers to confer towards positive outcomes. Patients can also specify that their data be sent to one or more providers in a file format that can be sent to the providers' email addresses.

According to various embodiments, as described in more detail below, the platform can be enabled by an app that uses a cloud presence to manage data, e.g. The app can sit on a desktop computer, laptop, tablet, and/or on a smartphone.

For research purposes, researchers are able to use the data housed under this platform to better understand behaviors and patient/doctor interactions by conducting deeper analysis beyond what is available in the mental health platform.

Privacy and security are built into this application, in compliance with such guidelines as HIPAA. For certain purposes, data can be anonymized/deanonymized through one of various methods as not to identify a specific patient who uses this application.

### I. Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the present invention are capable of being implemented in conjunction with any type of computing environment now known or later developed.

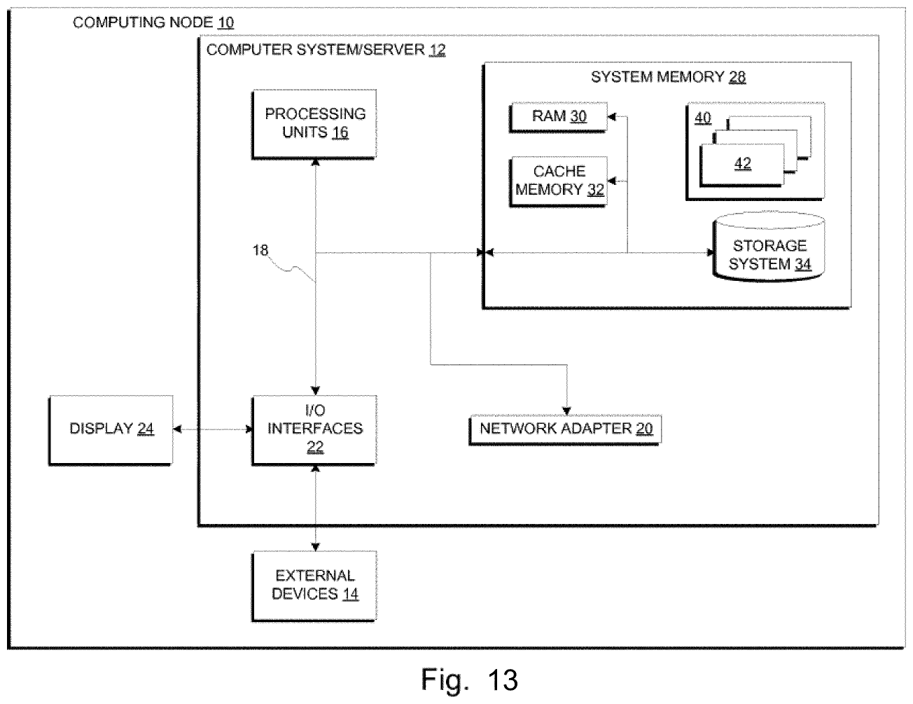

Referring now to FIG. 13, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, mobile devices, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 13, computer system/server 12 in computing node 10 is shown in the form of a general-purpose computing device. The components of computer system/server 12 may include, but are not limited to, processing units 16, a system memory 28, and a bus 18 that couples various system components including system memory 28 to processing units 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk, such as a CD-ROM, DVD-ROM or other optical media, can be provided. In such instances, each can be connected to bus 18 by one or more data media interfaces. As will be further depicted and described below, system memory 28 may include at least one program product having a set (e.g., at least one) of program modules that are configured to carry out the functions of embodiments of the invention.

Program/utility 40, having set of program modules 42, may be stored in system memory 28 by way of example, and not limitation, as well as an operating system, one or more application programs, other program modules, and program data. Each of the operating system, one or more application programs, other program modules, and program data or some combination thereof, may include an implementation of a networking environment. Set of program modules 42 generally carry out the functions and/or methodologies of embodiments of the invention as described herein.

Computer system/server 12 may also communicate with one or more external devices 14, such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks, such as a local area network (LAN), a general wide area network (WAN), and/or a public network (e.g., the Internet), via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

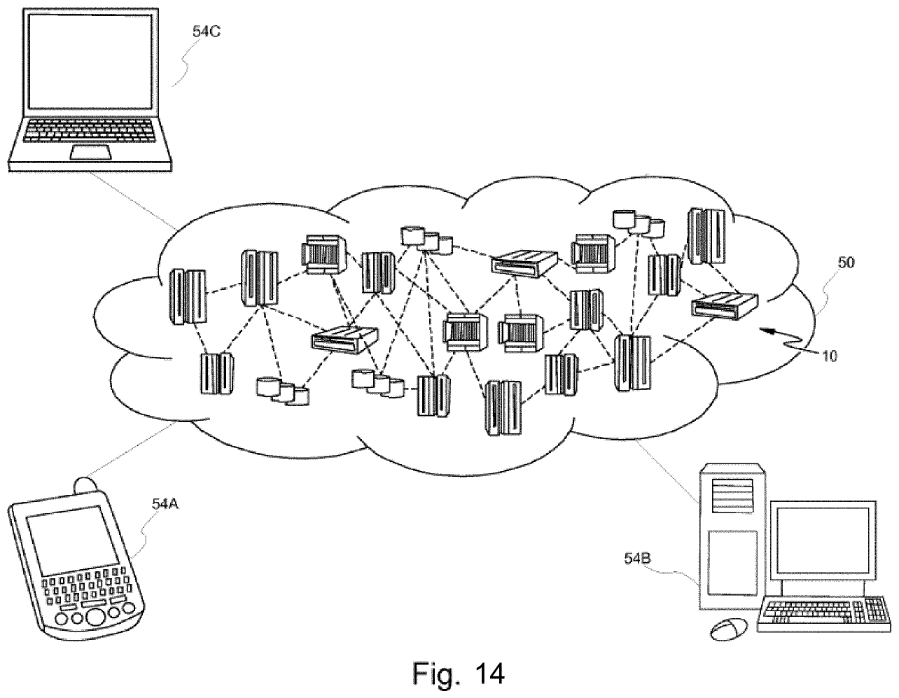

Referring now to FIG. 14, illustrative computing environment 50 is depicted. As shown, computing environment 50 comprises one or more computing nodes (e.g., computing node 10) with which local computing devices used by consumers, such as, for example, personal digital assistant (PDA) or cellular telephone 54A, desktop computer 54B, and/or laptop computer 54C may communicate. Computing nodes may communicate with one another. They may be grouped (not shown) physically or virtually, in one or more networks, such as Private, Community, Public, or Hybrid clouds as described hereinabove, or a combination thereof. This allows computing environment 50 to offer infrastructure, platforms and/or software as services for which a consumer does not need to maintain resources on a local computing device. It is understood that the types of computing devices 54A-C shown in FIG. 14 are intended to be illustrative only and that computing node 10 and computing environment 50 can communicate with any type of computerized device over any type of network and/or network addressable connection (e.g., using a web browser).

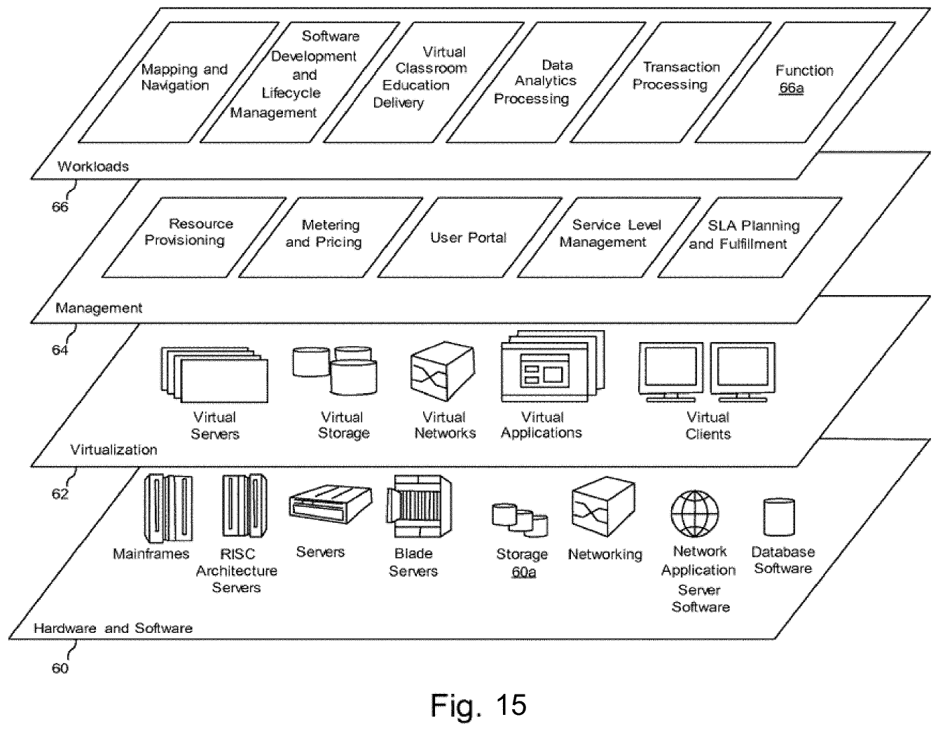

Referring now to FIG. 15, a set of functional abstraction layers provided by computing environment 50 (FIG. 14) is shown. It should be understood in advance that the components, layers, and functions shown in FIG. 15 are intended to be illustrative only and embodiments of the invention are not limited thereto. As depicted, the following layers and corresponding functions are provided:

Hardware and software layer 60 includes hardware and software components. Examples of hardware components include mainframes; RISC (Reduced Instruction Set Computer) architecture-based servers; storage devices; database software; networks, and networking components. In some embodiments software components include network application server software.

Virtualization layer 62 provides an abstraction layer from which the following examples of virtual entities may be provided: virtual servers; virtual storage; virtual networks, including virtual private networks; virtual applications and operating systems; and virtual clients.

In one example, management layer 64 may provide the functions described below. Resource provisioning provides dynamic procurement of computing resources and other resources that are utilized to perform tasks within the cloud computing environment. Metering and Pricing provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal provides access to the computing environment for consumers and system administrators. Service level management provides cloud computing resource allocation and management such that required service levels are met. Service Level Agreement (SLA) planning and fulfillment provide pre-arrangement for, and procurement of, computing resources for which a future requirement is anticipated in accordance with an SLA.

Workloads layer 66 provides examples of functionality for which the cloud computing environment may be utilized. Examples of workloads and functions which may be provided from this layer include: mapping and navigation; software development and lifecycle management; virtual classroom education delivery; data analytics processing; transaction processing; and functionality according to the present invention (see function block 66a) as will be discussed in detail, below, in the following sub-sections of this Detailed description section.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Accordingly, there is provided according to the invention, a computer system comprising:

a processor(s) set; a machine-readable storage device; and computer code stored on the machine readable storage device, with the computer code including instructions for causing the processor(s) set to perform operations including the following:
prompting a user to answer a series of questions directed to the user's state of health; storing said user's responses to said series of questions in a user health profile data set;
assessing said user's answers against a rule set to make one or more preliminary diagnoses and action recommendations;
communicate said preliminary and action recommendations to said user;
access a database of healthcare providers and make a recommendation to said user of one or more of said healthcare providers based on said one or more preliminary diagnoses;
initiate and manage online communications between said user and a selected healthcare provider;
   and
provide user and user-authorized healthcare provider access to said user information.

Additionally, the computer code of the invention may further include instructions for causing the processor(s) set to:
prompt the user to input or upload one or more of journal entries, diary entries, drawings, photographs, handwritten notes;
store user-inputted or uploaded documents in a user-accessible database;
process said user-inputted or uploaded documents against a ruleset as part of said preliminary diagnoses;
   and
   provide user-authorized healthcare provider access to user-inputted or uploaded documents from said user-accessible database;

According to further additional and/or alternative embodiments, the computer code of the invention may further include instructions for causing the processor(s) set to
make support group recommendations to a plurality of users;
introduce recommended support group users and facilitate
   and/or
manage communications between recommended support group users who accept system recommended support group participation.

Furthermore, the computer code of the invention may further include instructions for causing the processor(s) set to:
Generate chronologically-organized user mood and event timeline using user-input information
   and
present said chronologically-organized user-mood and event timeline to said user.

It will be appreciated by those skilled in the art that changes could be made to the preferred embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A computer system comprising:
a processor(s) set;
a machine-readable storage device; and
computer code stored on the machine readable storage device, with the computer code including instructions for causing the processor(s) set to perform operations including the following:
prompting a user to answer a series of questions directed to the user's state of health; storing said user's responses to said series of questions in a user health profile dataset;
assessing said user's answers against a rule set to make one or more preliminary diagnoses and action recommendations;
communicate said preliminary and action recommendations to said user;
access a database of healthcare providers and make a recommendation to said user of one or more of said healthcare providers based on said one or more preliminary diagnoses;
initiate and manage online communications between said user and a selected healthcare provider;
and provide user and user-authorized healthcare provider access to said user information.

2. The computer system of claim 1 wherein the computer code further includes instructions for causing the processor(s) set to perform the following operations:
prompt the user to input or upload one or more of journal entries, diary entries, drawings, photographs, handwritten notes;
store user-inputted or uploaded documents in a user-accessible database;
process said user-inputted or uploaded documents against a ruleset as part of said preliminary diagnoses;
provide user-authorized healthcare provider access to user-inputted or uploaded documents from said user-accessible database;

3. The computer system of claim 1 wherein the computer code further includes instructions for causing the processor(s) set to perform the following operations:
make support group recommendations to a plurality of users;
introduce recommended support group users and facilitate and/or manage communications between recommended support group users who accept system recommended support group participation.

4. The computer system of claim 1 wherein the computer code further includes instructions for causing the processor(s) set to perform the following operations:
Generate chronologically-organized user mood and event timeline using user-input information and present said chronologically-organized user-mood and event timeline to said user.
